(19) United States
(12) Patent Application Publication
(10) Pub. No.: US 2015/0032068 A1
(43) Pub. Date: Jan. 29, 2015

(54) Adjustable Compression Garment System And Method

(71) Applicant: Janice Trane Jones, Peoria, AZ (US)

(72) Inventor: Janice Trane Jones, Peoria, AZ (US)

(21) Appl. No.: 14/340,528

(22) Filed: Jul. 24, 2014

Related U.S. Application Data
(60) Provisional application No. 61/858,041, filed on Jul. 24, 2013.
(60) Provisional application No. 62/002,852, filed on May 24, 2014.

(51) Int. Cl. A41C 3/00; A61F 5/44

(52) U.S. Cl. CPC A41C 3/0028; A41C 3/0064; A61F 5/44
USPC 604/327; 450/86

(57) ABSTRACT

A modular and highly adaptable and adjustable compression garment system is provided. The system can include a garment body portion, shoulder strap portions, side portions, a back portion, and one or more modular strap adjustment systems. The garment body portion can include attachment substrates, rail devices, loop devices, and like attachment configurations.

### RELATED APPLICATIONS

This application claims priority to and the benefit of U.S. Provisional Application No. 61/858,041, filed Jul. 24, 2013, and U.S. Provisional Application No. 62/002,852, filed May 24, 2014, each of which is incorporated fully herein by reference.

### FIELD OF THE INVENTION

The present invention generally relates to post-surgical garments. More particularly, the present invention relates to compression garments to provide support and contouring for patients after breast surgeries or similar procedures, including breast implant, reconstruction, or enhancement surgeries.

### BACKGROUND OF THE INVENTION

Conventional attempts to provide garments for patients after breast surgery, e.g., breast augmentation, lifts, or reconstruction, are generally quite functionally limited. The garments are typically simple bras or compression bands that are adapted to surround the breasts and extend around the upper torso to provide some level of implant positioning and stabilization.

However, these conventional garments are not designed to be worn immediately after surgery as they provide very inexact compression benefits. Further, patients are either stuck with a one-size-fits-all garment that is not adapted to properly support and stabilize the unique anatomical features of the patient, or are forced to purchase multiple possible garments in an attempt to find the one that will properly address the needs of that patient, at that time. The various needs for a patient after a particular procedure can vary greatly, and those needs often change over time as problems arise or even as the normal healing process progresses. Current garments are not equipped to handle so many different patient needs. As a result of these single use or generally non-configurable conventional garments, many patients after breast surgery are unable to obtain desirable support to advance proper healing and treatment. And these deficiencies can be both physically and emotionally challenging.

### SUMMARY OF THE INVENTION

The present invention addresses certain deficiencies discussed above by providing a modular and highly adaptable and adjustable compression garment system to promote treatment during the entire healing and implant stabilization process.

The system can include a garment body portion, shoulder strap portions, side portions, a back portion, and one or more modular strap adjustment systems. Portions of the system can be constructed of elastic or semi-elastic materials or cloth to facilitate stretching and adjustment around the body of the patient, while other portions of the system can be constructed of a generally non-elastic but comfortable material to advance consistent support and securement properties. Further, the system can come in a plurality of different base sizes (e.g., XS, S, M, L, XL, etc.).

The one or more modular strap adjustment systems can include a plurality of modular adjustment means to adapt to the unique anatomical and support needs of a particular patient. For instance, various embodiments of the present invention can include attachment portions or clip rail segments surrounding all or a portion of a central garment body portion including a first breast region and/or a second breast region. The garment system can be divided and constructed such that it fits on the patient much like a shirt or vest.

The adjustment systems can further include strap members. The strap members can be constructed of various known materials and are generally adapted to operatively attach or secure to the attachment portions or rail segments via one or more clip devices. The strap members can be defined by a width generally equal to the length of the segments, can be wider than the segments, narrower than the segments, or can include other undulations, tapers, features, shapes and sizes as desired for a particular application or use to provide desirable adjustability and treatment flexibility for a given patient or need.

This garment can be selected by either the surgeon or patient to be worn immediately after surgery and throughout the recovery process for augmentations as well as reconstructive breast surgeries. A surgeon may choose to add or remove strapping at any or all milestones of the recovery process. The surgeon can make any and all strapping adjustments with available purchased straps, clips and the like. The compression garment is comfortable and provides physical and emotional support. Women going through mastectomies, augmentations and other breast surgeries will have trepidation and fears and the present garment system promotes the feeling of safety and well being as a result of the uniquely and individually tailored design and treatment.

A fluid drainage bag or system can be included, which can be easily cleaned and/or removed with minimal effort by the patient while still following the physician's instructions.

The front clasping and highly adjustable garment of the present invention can be adjusted and tensioned easily by the patient without additional assistance or unnecessary visits to the surgeon's office.

Embodiments of the present invention can include one or more, and generally a plurality of, attachment regions or substrates adapted to provide adjustability and securement for the garment. Regions of the garment can be constructed of a stretch or non-stretch material, such as a cotton or cotton/poly blend, and can include substrates or other attachment devices, structures, or mechanisms. Other materials for portions of the garment can include high grade compression lycra, spandex/chinlon, and soft stretch lace nylon/spandex.

The substrates can be included on a top or edge surface of the regions, or integrated or otherwise provided under a layer of the material such that fastening sections are accessible for fixation and modular adjustability. The substrates can be constructed of a rigid, semi-rigid or generally flexible material, such as vinyl, PVC or like polymer or metal materials. The regions can further include loop devices, wherein clips at the end of adjustment straps are selectively engaged with the loop devices to provide the desired modular adjustability.

The materials, perforations, rails, substrates, loop devices and other devices or mechanism disclosed herein can be employed at any portion of the garment (e.g., front, back, sides, etc.) and can assume the construct of any of the disclosure herein without deviating from the spirit and scope of the present invention. Further, the portions, loops, perforations, rails, substrates, and the like can be constructed of rigid, semi-rigid, flexible, and semi-flexible materials as desired for embodiments and particular applications of the present invention.

### BRIEF DESCRIPTION OF THE DRAWINGS

FIG. 1 shows a plan view of a compression garment in accordance with embodiments of the present invention.

FIGS. 2-3 are perspective views of a compression garment being worn by a user in accordance with embodiments of the present invention.

FIG. 4 is a view of the back of a compression garment being worn by a user in accordance with embodiments of the present invention.

FIG. 5 is a plan view of connection segments for a compression garment in accordance with embodiments of the present invention.

FIG. 6 is a perspective view of connection segments for a compression garment in accordance with embodiments of the present invention.

FIGS. 7-8 are side views of a clip device, and its use, in accordance with embodiments of the present invention.

FIG. 9 is a plan view of connection segments for a compression garment in accordance with embodiments of the present invention.

FIGS. 10-11 are perspective views of clip devices engaging with corresponding connection segments for a compression garment in accordance with embodiments of the present invention.

FIGS. 12-13 are side views of exemplary clip devices, and their use, in accordance with embodiments of the present invention.

FIGS. 14-15 are side views of exemplary clip devices, and their use, in accordance with embodiments of the present invention.

FIG. 16 is a top view of a tension strap including a clip device, for a compression garment, in accordance with embodiments of the present invention.

FIG. 17 is a perspective view of a tension strap including a clip device, and an opposing clip device, in accordance with embodiments of the present invention.

FIG. 18 is a schematic view of two opposing interlocking members to selectively close and open a compression garment, in accordance with embodiments of the present invention.

FIG. 19 is a schematic view of two opposing and interlocked members in use to selectively close a compression garment, in accordance with embodiments of the present invention.

FIGS. 20-22 are views of a drainage bag system for use with a compression garment in accordance with embodiments of the present invention.

FIGS. 22a-22b are views of a drainage bag holder system for use with a compression garment in accordance with embodiments of the present invention.

FIGS. 23a-23e are views of a compression garment, and components, with perforated substrates in accordance with embodiments of the present invention.

FIG. 24 is a view of a breast region of a compression garment with perforated substrates in accordance with embodiments of the present invention.

FIG. 25 is a view of a breast region of a compression garment with rail attachment devices in accordance with embodiments of the present invention.

FIG. 25a is a close-up isolated view of a rail attachment device in accordance with embodiments of the present invention.

FIG. 26 is a front view of a compression garment having substrate rail attachment devices in accordance with embodiments of the present invention.

FIG. 27 is a side view of a compression garment having substrate rail attachment devices in accordance with embodiments of the present invention.

FIG. 28 is a back view of the compression garment of FIG. 26.

FIGS. 29a-29b are views of straps and clips for use with a compression garment having substrate rail attachment devices in accordance with embodiments of the present invention.

FIGS. 30-32 show views of rail attachments for use with a compression garment in accordance with embodiments of the present invention.

FIG. 33 is a front view of a compression garment having loop attachment devices in accordance with embodiments of the present invention.

FIG. 34 is a back view of the compression garment of FIG. 33.

FIG. 35 is a side view of a compression garment having loop attachment devices in accordance with embodiments of the present invention.

FIGS. 36-37 are views of strap and clip devices and mechanisms for use with a compression garment having loop attachment devices in accordance with embodiments of the present invention.

While the invention is amenable to various modifications and alternative forms, specifics thereof have been shown by way of example in the drawings and will be described in detail. It should be understood, however, that the intention is not to limit the invention to the particular example embodiments described. On the contrary, the invention is to cover all modifications, equivalents, and alternatives falling within the scope of the invention as defined by the appended claims.

### DETAILED DESCRIPTION OF PREFERRED EMBODIMENTS

In the following description, the present invention will be explained with reference to various exemplary embodiments. Nevertheless, these embodiments are not intended to limit the present invention to any specific example, environment, application, or particular implementation described herein. Therefore, descriptions of these example embodiments are only provided for purpose of illustration rather than to limit the present invention.

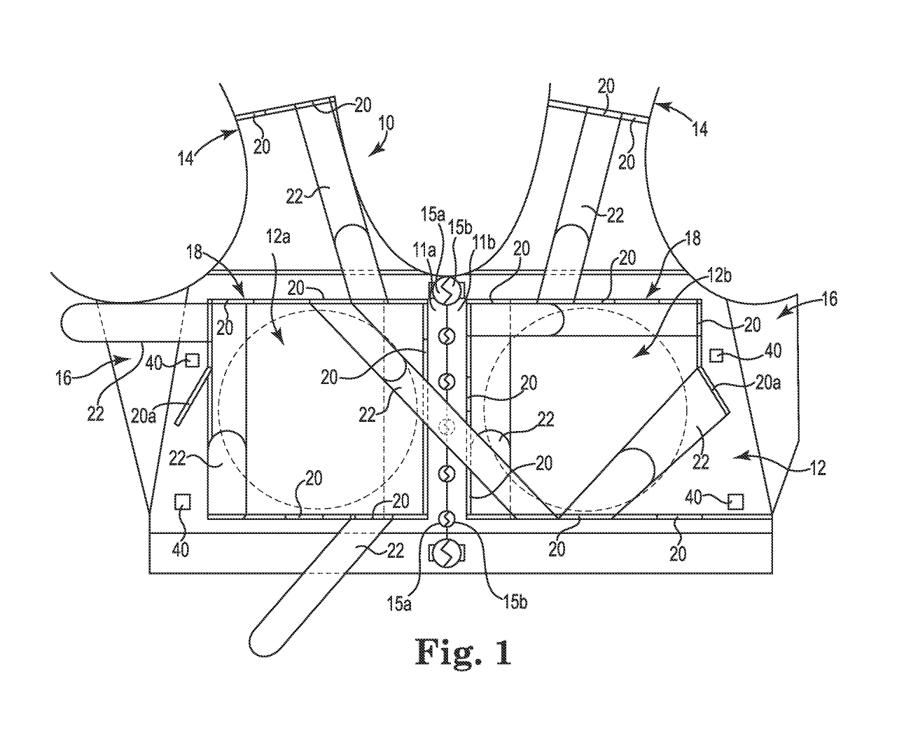

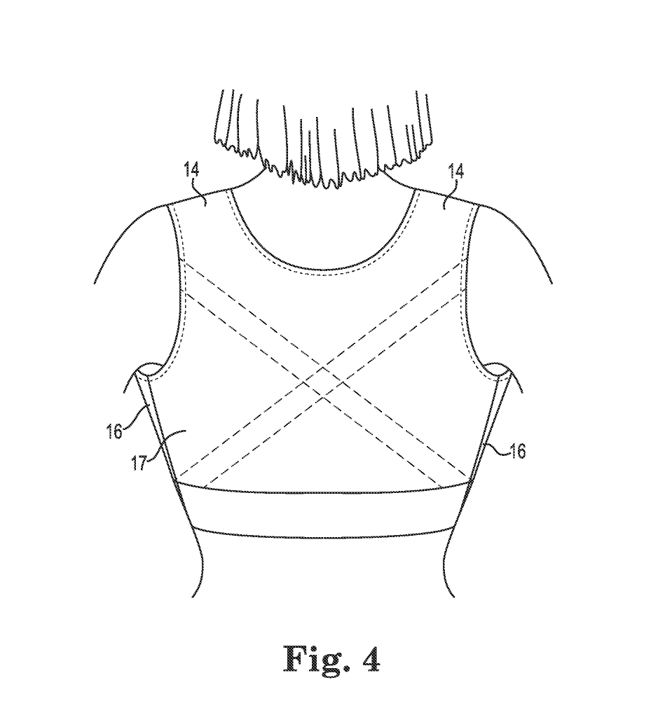

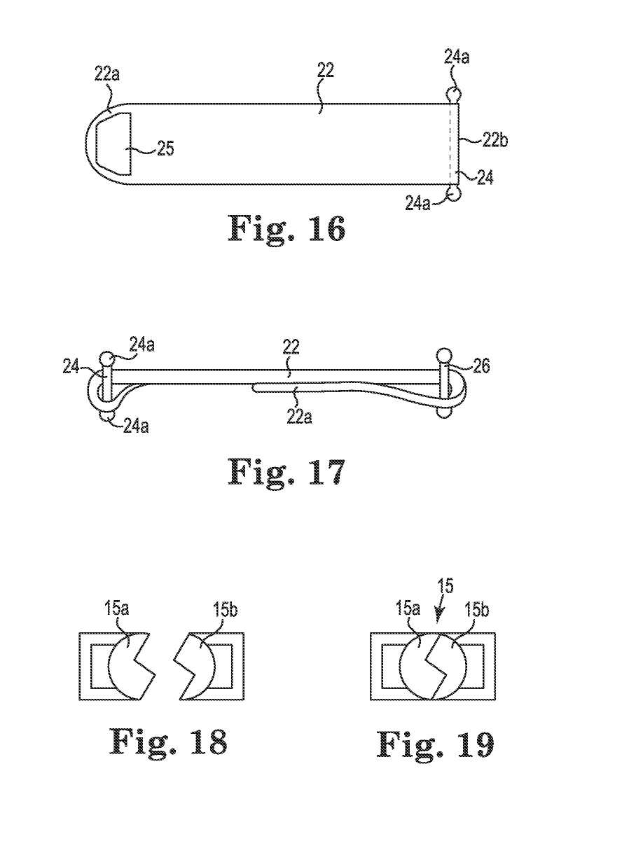

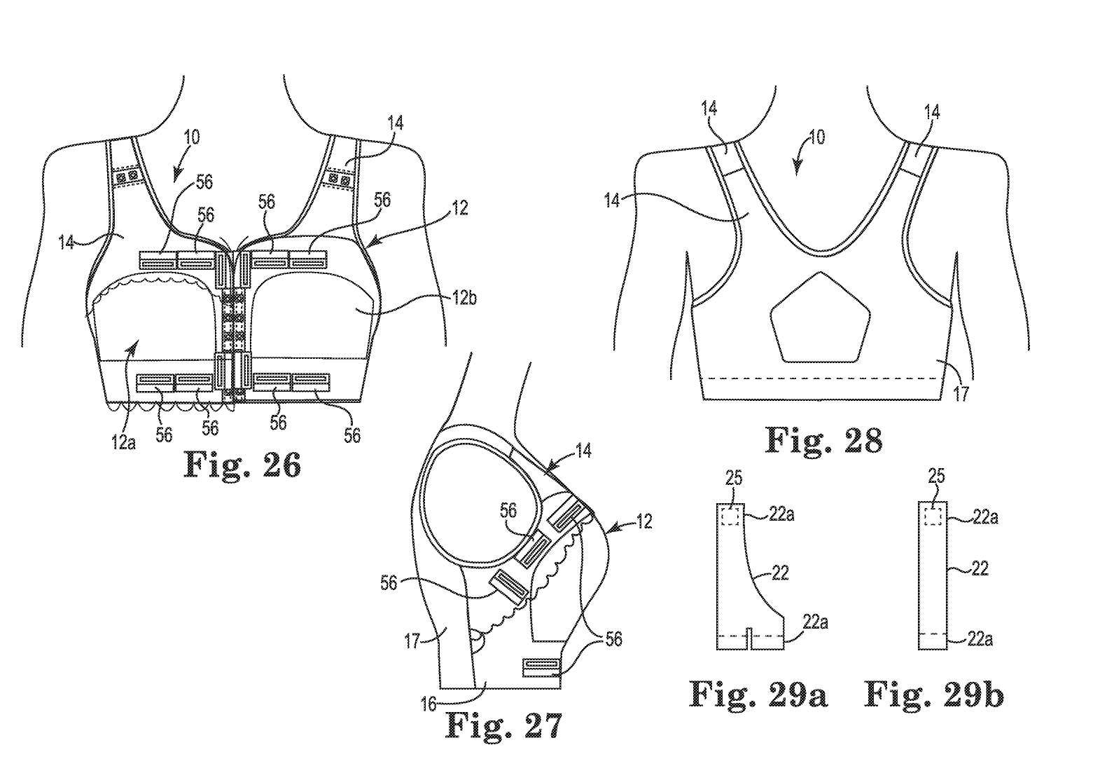

Referring to FIGS. 1-37, a compression garment or bra system 10 is disclosed. The system 10 is adapted for a patient to wear after breast surgery, including breast augmentation, reconstruction or like procedures. Other situations or treatment opportunities are also envisioned for the present invention. In general, the patient will receive implants during these procedures, and post-operative support and stabilization of the implants is typically required. However, the size of the implants, the degree of tissue trauma, the size of the patient's upper torso or chest region, and other unique anatomical dimensions and proportions of the patient's body should be considered. The healing or treatment process can vary depending on the particular timeframe or stage the patient's body is at in the process, and the system 10 of the present invention is modular and highly adaptable and adjustable to account for such factors such that a single system, or a set of systems, provides desirable support and treatment during the entire healing and stabilization process.

The system 10 can include a garment body portion 12, shoulder or strap portions 14, side portions 16, a back portion 17, and one or more modular strap adjustment systems 18. Portions of the system 10 can be constructed of elastic or semi-elastic materials or cloth to facilitate stretching and adjustment around the body of the patient, while other portions of the system 10 can be constructed of a generally non-elastic but comfortable material to advance consistent support and securement properties. Materials for forming portions of the garment 10 can include high grade compression lycra, spandex/chinlon, and soft stretch lace nylon/spandex. Further, the system 10 can come in a plurality of different base sizes (e.g., XS, S, M, L, XL, XXL, etc.).

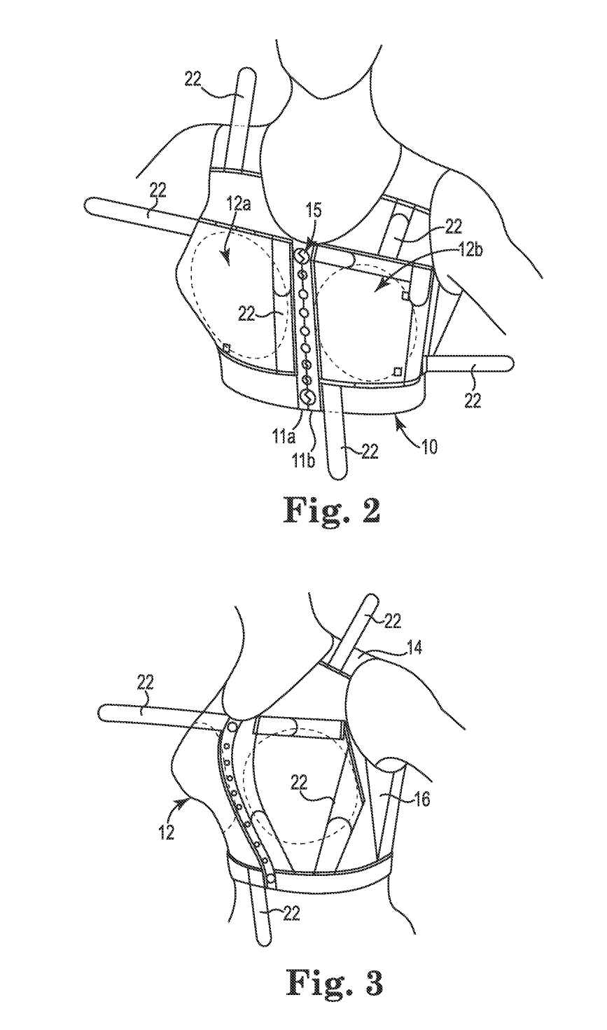

Referring generally to FIGS. 1-3, the one or more modular strap adjustment systems 18 are shown. The systems 10 can include a plurality of modular adjustment means to adapt to the unique anatomical and support needs of a particular patient. For instance, various embodiments of the present invention can include attachment portions or clip rail segments 20 surrounding all or a portion of a central garment body portion including a first breast region 12a and a second breast region 12b. FIG. 1 depicts circular dashed line regions to demonstrate where the patient's breasts can be positioned within the respective regions 12a, 12b upon use. FIGS. 2-3 further depict the system 10 in use with a patient. The garment system 10 can be divided and constructed such that it fits on the patient much like a shirt or vest, with each region 12a, 12b provided on opposing front portions of the vest. For instance, as shown in FIGS. 1-3, each separable region of the garment can include a plurality of vertically aligned clips, buttons, flaps, snaps, a zipper, or like devices, techniques or mechanisms 15 to selectively and releasably close or open up the garment for fitting to the patient, or removing from the patient.

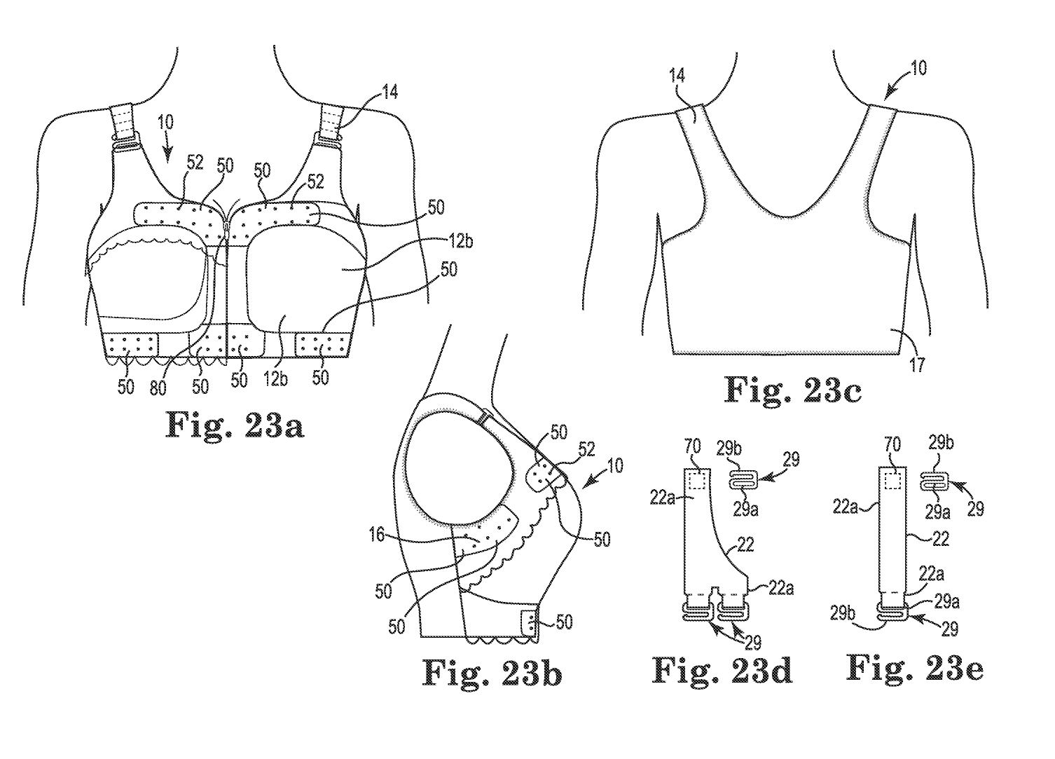

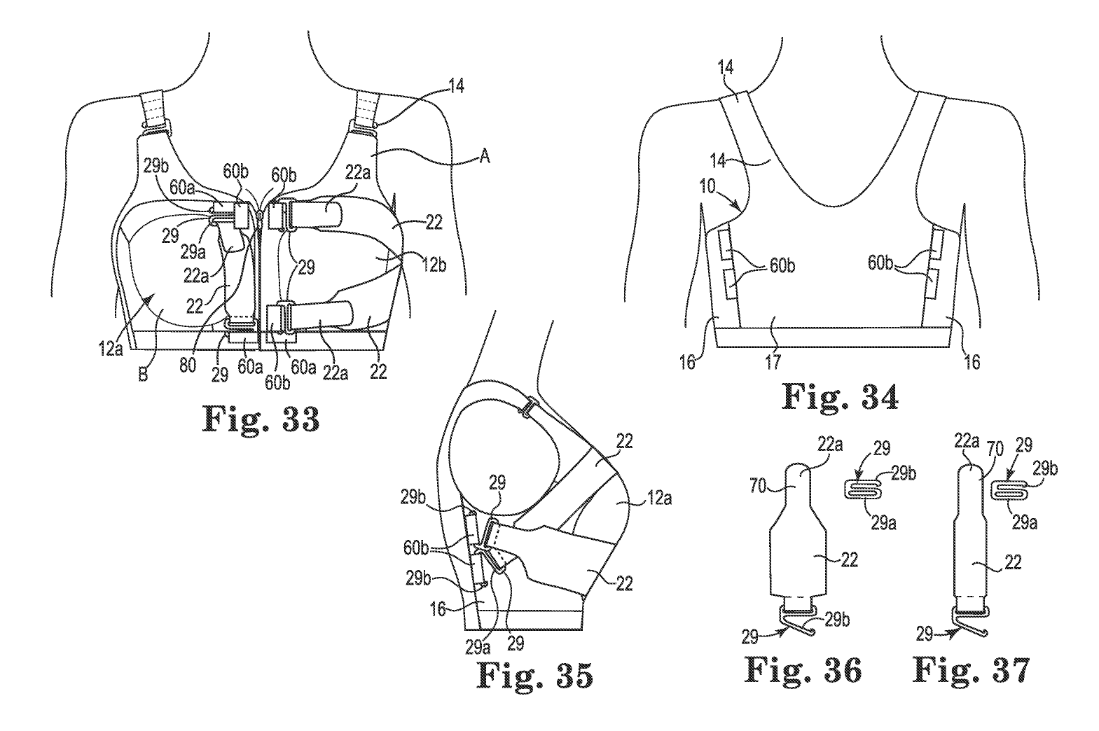

In certain embodiments, the devices 15 include opposing but securely matable interlocking members 15a, 15b, as shown in FIGS. 18-19. A plurality of the interlocking members 15a can be provided along a seam or side 11a of the garment, while a plurality of the interlocking members 15b can be provided along a seam or side 11b of the garment such that snapping or like engagement of the members 15a, 15b generally secures the garment in place around the upper body of the patient, as shown in FIGS. 2-3. Other embodiments disclosed herein can include a zipper or like recloseable access device or mechanism to open, close or secure the garment in place. For instance, the embodiments of FIGS. 23a and 33, include a zipper device 80 to selectively join the breast regions together.

The adjustment systems 18 can include strap members 22. The strap members 22 can be constructed of various known materials and are generally adapted to operatively attached or secure to the attachment portions or rail segments 20 via one or more clip devices 24. The strap members 22 can be defined by a width generally equal to the length of the segments 20, can be wider than the segments, narrower than the segments, or can include other undulations, tapers, features, shapes and sizes as desired for a particular application or use. The straps 22 can come in various lengths (e.g., 8″, 12″, and the like) and various sized straps 22 can be provided with a particular system 10 to provide optimal adjustability and flexibility for a given patient or treatment need.

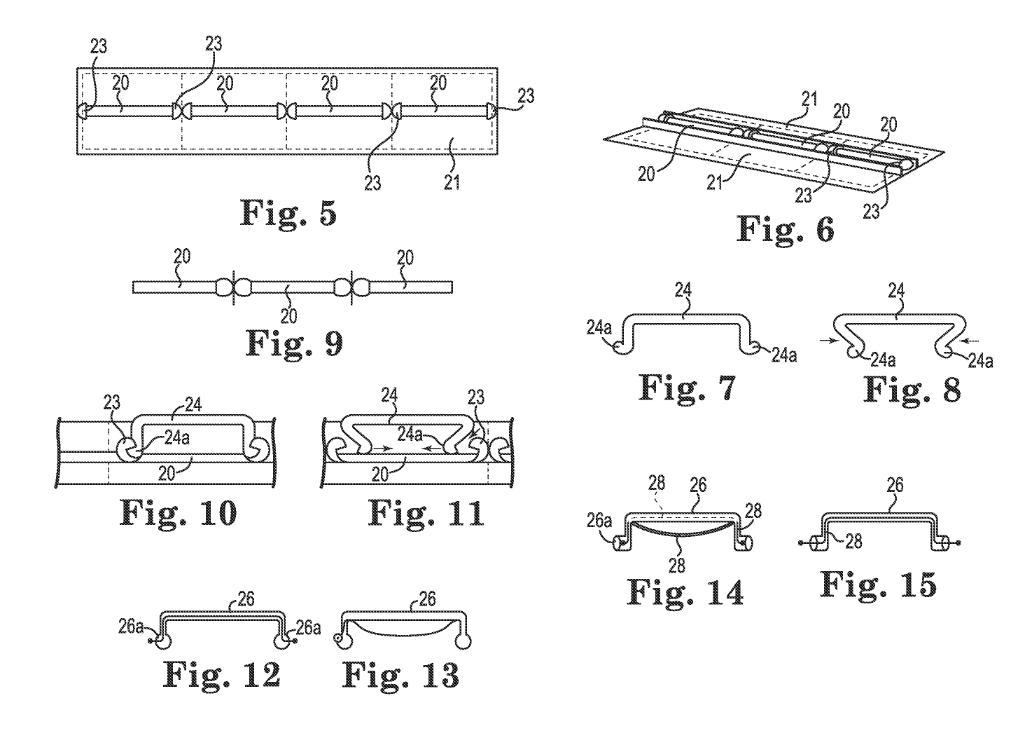

The rail segments 20 can be provided with the present invention in various configurations and designs. For instance, the length of each segment 20 can be approximately 1″-2″ in certain embodiments and can be adapted to accommodate or securely mate with a corresponding clip device 24. However, devices and mechanisms having other structural, connectivity, and dimensional characteristics are envisioned for the present invention as well, consistent with the teachings provided herein. The plurality of serial segments 20 can be provided directly to the garment material, provided along a separate rail or seam member/material 21 generally having increased reinforcement characteristics (as shown in FIGS. 5-6), or in a variety of other ways as is apparent from the present disclosure. As depicted in FIG. 6, certain embodiments of the segments 20 can take on a groove or open channel construct to facilitate connectivity with corresponding devices 24.

Again referring to FIG. 1, the rail segments 20 can be provided with the garment body 12, around the periphery of the breast regions 12a, 12b. Certain segments 20 can be included serially along linear, angled, arcuate or other like lengths, and in various embodiments can include lengths of multiple segments 20 disposed in a generally transverse or perpendicular orientation. Other segments, or a plurality of segments, can extend out from various angles relative to other or adjacent segments, such as shown with segments 20a. Selective placement and use of these segments for the system 10 provides highly targeted adjustability for tensioning the garment at desired regions to facilitate personalized and focused implant support and stabilization. The patient can work with a physician after the surgical procedure to attach and tension straps to specific segments 20 such that only those segments 20 and straps 22 needed to achieve the tensioning support, stabilization, angles and optimal positioning are used. Similarly, the patient or the patient's physician can later adjust the system by using different straps or by positioning and securing the straps at different areas or along different tensioning paths between opposing segments 20, as needed.

FIGS. 7-15 show various embodiments of the clip devices 24, or clip devices 26, and interlocking connectivity with respective segments 20. In certain embodiments, the straps 22 can include a free end 22a and a clip device 24 provided (e.g., integrally or separately attached) at an end opposite the free end 22a. The clip device 24, and other embodiments or versions of clip devices disclosed herein, can include end bulbous 24a or like features adapted to mate with corresponding matable features in the segments 20. As shown in FIGS. 7-8, the devices 24 can be constructed of compatible polymer or metal materials adapted to measurably bend or “give,” and release relative to the target segments 20. Similarly, the segments 20 can include end features 23 adapted to at least partially shroud or otherwise engage with the features 24a of the devices 24 (e.g., FIGS. 5-6) to facilitate secure locking or engagement.

As depicted in FIGS. 10-11, a device 24 (e.g., at the end of a strap 22) is shown selectively engaging and releasing from a target segment 20. A free or isolated clip device 26, as shown in FIGS. 12-15, can be engaged with a segment 20 adapted to receive the free end 22a of the strap 22. As such, the clip device 24 can be inserted into selective engagement with a designated segment 20, with the clip device 26 selectively engaged with a segment 20 linearly or angularly opposing the clip device 24 such that the free end of the strap 22 is adapted to extend through and/or around the isolated clip device 26 to allow for highly targeted tensioning, compression and/or support across a unique strap path (e.g., linear or angular). In various embodiments, the free end 22a of the strap 22 can include a hook and loop fastener (e.g., Velcro®), clips, fasteners, snaps, or like engagement features 25 to retain the free end 22a securely in place to maintain the desired tensioning and support across the strap path.

Certain embodiments of the isolated clip device 26, as shown in FIGS. 12-15, can include an interior lumen having a wire or like member 28 disposed therein and extending out from the bulbous end portions 26a. As such, in a relaxed state for the clip device 26, the ends of the member 28 generally reside within the lumen of the device 26. As the device 26 is pinched or otherwise deformed, the ends of the member 28 can protrude from the end portions 26a of the device 26 for selective engagement with the receiving portions 23 of the target segment 20. Again, adjustable tensioning is provided by selecting and correspondingly engaging straps 22 across therapeutically targeted and distally opposed segments 20 to define optimal tensioning and stabilization paths.

In various other embodiments, both ends of the strap 22 can include integrated clip devices 24, both ends of the strap 22 can be free ends adapted to engage separately placed clip devices 24, or the strap and clip device design can assume variations and modifications of the expressly depicted configuration such that the targeted and highly adjustable nature of the present invention is promoted as disclosed herein.

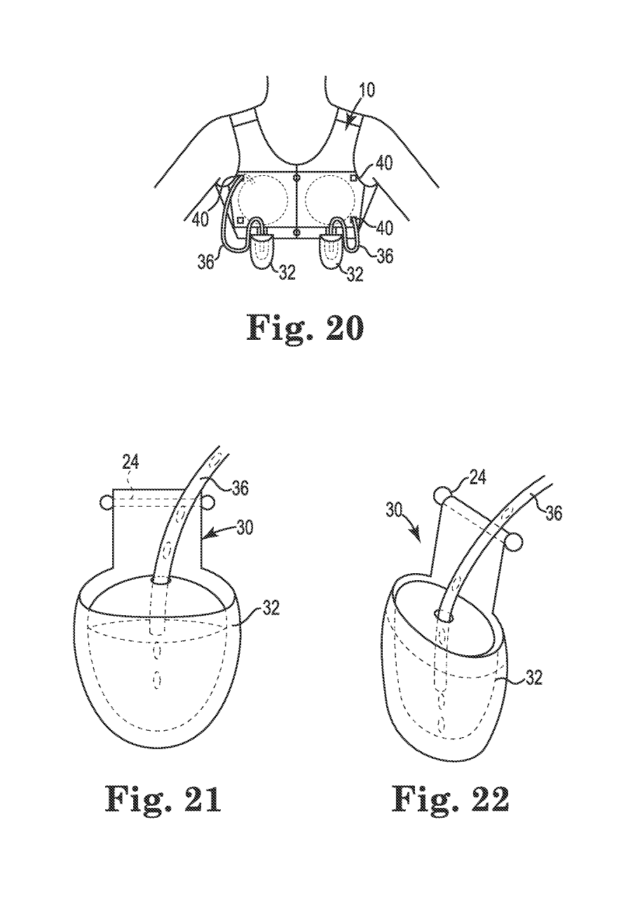

FIGS. 20-22b show embodiments of the garment system 10 including one or more fluid drainage device or bag systems 30. The bag systems 30 can include a reservoir portion 32, an attachment portion 34, and one or more fluid conduits 36. The garment can include one or more apertures or fluid access openings 40 adapted to receive the fluid conduit 36 of the bag systems 30. One end of the fluid conduit 26 is in communication with the reservoir portion 32, while the distal end can be surgically or otherwise engaged with an incision or like opening into the tissue of the patient, such that fluid from the surgical site can be expelled out through the conduit 26 and into the externally disposed reservoir portion 32 of the bag system 30. Like the clip devices 24 and/or 26, the attachment portion 34 of the bag system 30 can be constructed as a releasable/compressible clip adapted to selectively engage with a respective segment 20. As such, one or more reservoir portions 32 can be optimally located along a portion of the system 10. The reservoir portion 32 can be appropriately sealed to prevent fluid draining from the surgical site from exiting once it has entered via the conduit 36. One or all of the components of the bag system 30 can be periodically replaced or repositioned in accordance with the treatment needs of the patient.

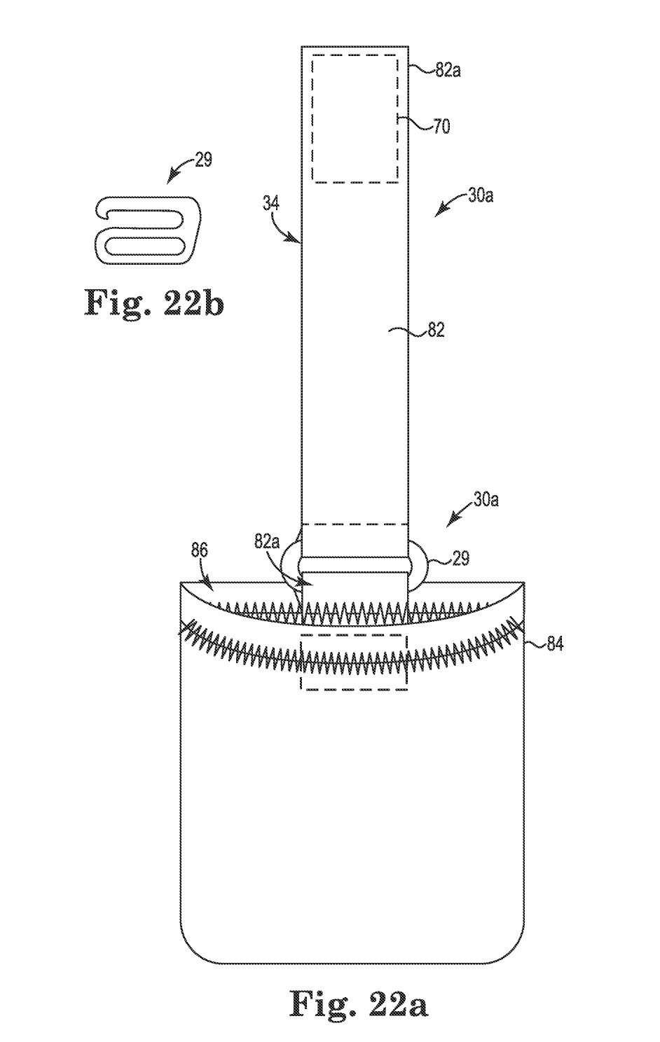

FIGS. 22a-22b, show an embodiment of a drainage bag holder 30a for use with the present invention. One or more components of the bag system 30 can be incorporated into or placed within the bag holder. The bag holder 30a can include a strap 82 having strap free ends 82a, and a holding portion 84, with the holding portion 84 including an internal compartment 86 adapted to receive various components, including the reservoir portion 32 and a portion of the conduit 36. Like other embodiments disclosed herein, one or more clips 29 can be included to attach to the free ends 82a such that the holder 30a can be selectively positioned along various portions of the garment 10 such as loop devices, attachment rails, substrates, and the like. For those embodiments employing attachment loops, at least one free end 82a can include hook and loop fasteners or like devices or mechanisms to thread through or otherwise secure with the clips 29.

FIGS. 23a-32 show embodiments of the present invention including one or more, and generally a plurality of, substrates 50 adapted to provide adjustability and securement for the garment 10. The regions 12a, 12b, 16 can be constructed of a stretch or non-stretch material, such as a cotton or cotton/poly blend, high grade compression lycra, spandex/chinlon, and soft stretch lace nylon/spandex, and can include the substrates 50. The substrates 50 can be included on a top surface of the regions, or integrated or otherwise provided under a layer of the material such that fastening sections are accessible for fixation and modular adjustability. The substrates 50 can be constructed of a rigid, semi-rigid or generally flexible material, such as vinyl, PVC or like polymer or metal materials.

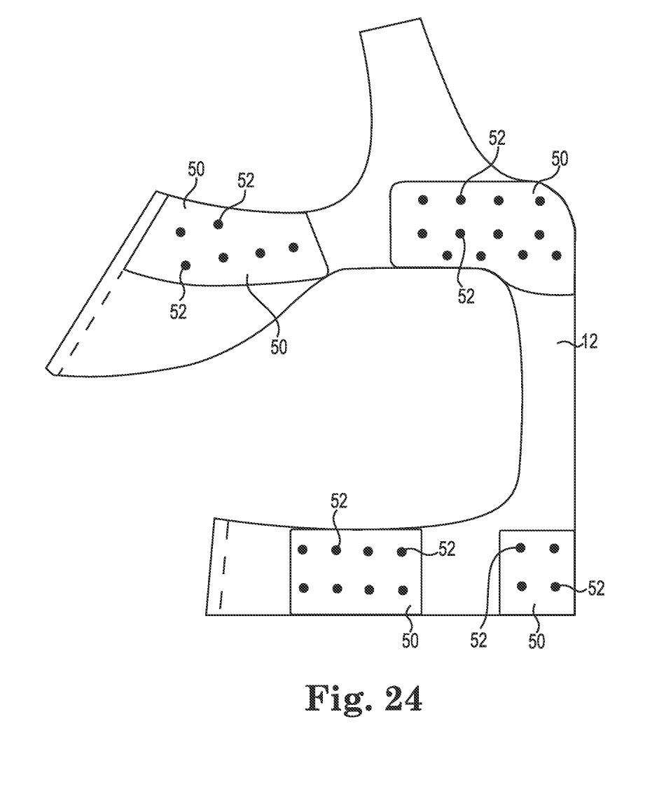

As shown in FIGS. 23a-24, the substrates 50 can include a plurality of perforations 52 or like apertures or features configured to receive one or more straps 22, clips, members, protrusions, and the like as disclosed in detail herein. The free ends 22a of the straps 22 can can be extended or threaded through an aperture 29a of a clip device 29. The clip device 29 can be constructed of various materials, including plastic or metals. The clip device 29 can further include an arm or member 29b adapted to selectively engage with the substrate 50—e.g., target perforations 52—to facilitate the highly adjustable nature of the present invention. In certain embodiments, a bra adjuster is clipped through the substrate as needed to provide this selective adjustability.

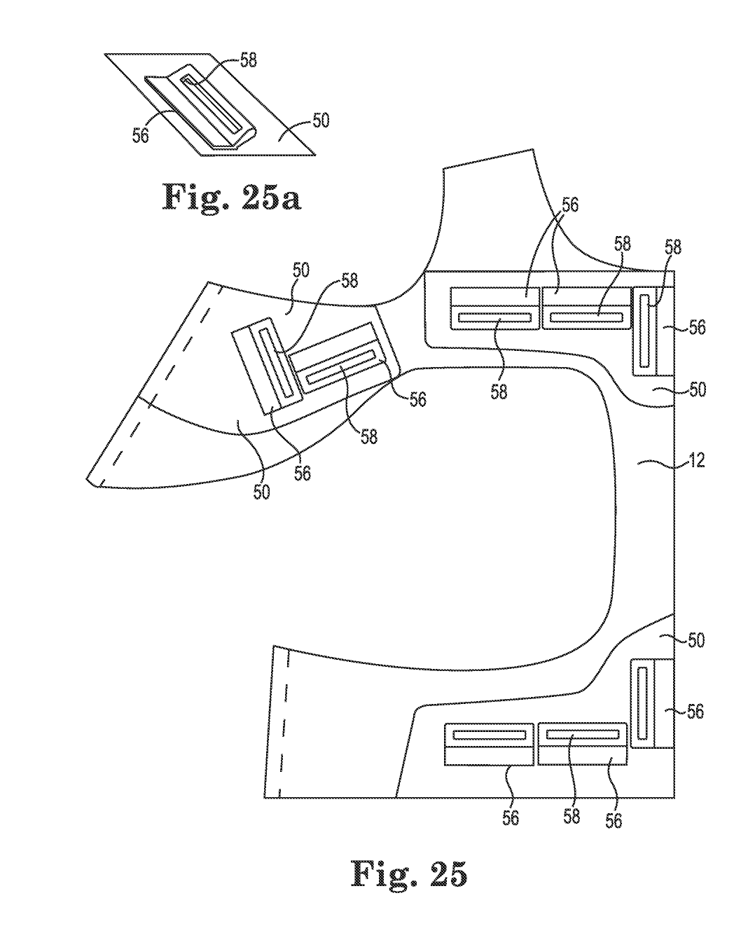

In various embodiments, such as those depicted in FIGS. 25-32, the substrates 50 can include one or more clips or like rail devices 56 welded/molded to or otherwise provided with the substrates 50 to facilitate attachment. The devices 56 can be of different widths and lengths (e.g., 1″-5″), even when provided on the same breast region. The devices 56 can include an elongate aperture or recess 58 adapted to receive the straps 22, or ends 22a, for adjustable securement. As detailed herein, the devices 56 can be provided at various angles and locations along the garment 10 to permit numerous levels of selective tensioning and pressure upon the patient's breasts. The free ends of the straps 22 can include hook and loop fasteners (FIGS. 29a-29b) in certain embodiments and can be fed through the apertures 58 and fixated therein. One or more ends 22a of a particular strap 22 can include two distinct portions adapted to engage two adjacent devices 56 (FIG. 29a).

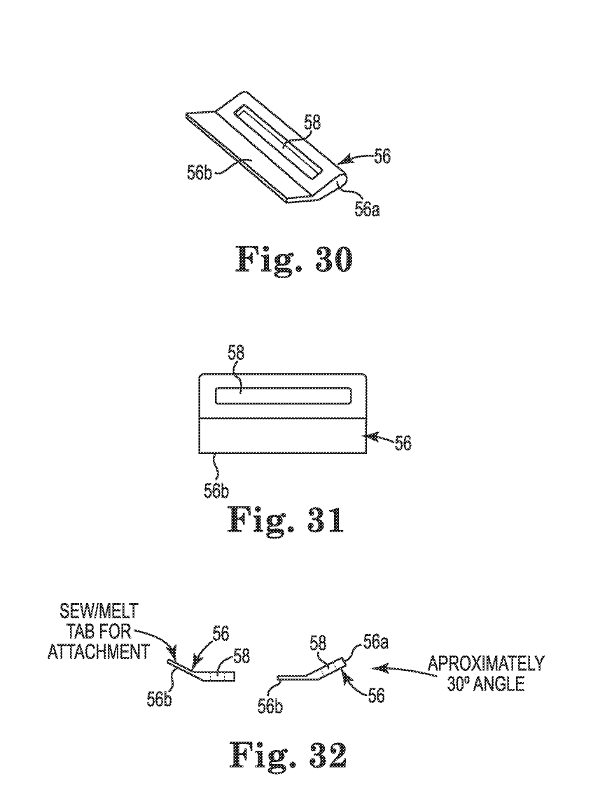

As shown in FIGS. 30-32, the rail devices 56 can include an angled portion 56a and a generally flat tab portion 56b. As such, one or more portions of the devices 56 can be angled and positioned for improved fixation. The tab portion 56b can be sewn, melded, or otherwise provided with a portion of the garment 10. Further, the figures show various angles, materials, dimensions and overall construction configurations for embodiments of the devices 56. However, a myriad of shapes, sizes and constructs can be implemented for the main portion 12, the substrates 50, the features 52, and the devices 56 without deviating from the spirit and scope of the present invention. Selective placement and use of these substrate attachment regions provides highly targeted adjustability for tensioning the garment at desired regions to facilitate personalized and focused implant support and stabilization. The patient can work with a physician after the surgical procedure to attach and tension straps to specific substrates such that only those substrates, or portions thereof, needed to achieve the tensioning support, stabilization, angles and optimal positioning are used. Similarly, the patient or the patient's physician can later adjust the system by using different straps or by positioning and securing the straps at different areas or along different tensioning paths as needed.

FIGS. 33-37 depict an embodiment of the present invention including one or more loop devices (e.g., constructed of fabric) or mechanisms 60 to facilitate selective adjustment of the garment 10. The devices 60 can be used in lieu of the substrates 50 or rails 20, or in conjunction therewith. One or more edge portions 62 of the devices 60 can be sewn directly into the foundation area or similar seams of the garment 10 for a more comfortable and structured positioning and adjustment system. As such, the patient can wear the garment both day and night with less discomfort. Further, such embodiments can provide a lighter and cooler garment to promote comfort and wearability. Embodiments of the garment 10 can include a base material A and a lace material B. The lace material B and other components and materials can be overlaid onto the base material A to construct the garment 10.

The loop devices 60 can include generally horizontal loop devices 60a or generally vertical loop devices 60b. The various loop devices can be constructed of an elastic material. In addition, generally vertical side loop devices 60c can be included at one or both of the side regions 16 of the garment 10. In certain embodiments, as shown in FIG. 33, the generally horizontal loop devices 60a can be opposingly positioned at one or both of the breast regions 12a, 12b such that one of the straps 22 can adjustably extend therebetween in a generally vertical or vertically angular orientation. The generally vertical loop devices 60b can also be opposingly positioned at one or both of the breast regions 12a, 12b, with one of the straps 22 extending between one of the vertical loop devices 60b and one of the vertical side loop devices 60c. As depicted in FIG. 35, straps 22 connecting to the side loop devices 60c can overlap or crossover one another to generally provide support for the corresponding breast region of the garment 10. While FIG. 33 depicts the first breast region 12a having a single generally vertical strap 22, this is for illustrative purposes only, and one or more additional straps can be connected to and extend from the vertical loop devices 60b to the side loop devices 60c. Similarly, the second breast region 12b can include one or more straps connecting to and extending across opposing horizontal loop devices 60a of the second breast region 12b, in lieu of or in conjunction with the straps extending between the vertical loop devices 60b and the side loop devices 60c. Furthermore, each of the regions 16a, 16b, 16c can include multiple loop devices 60a, 60b (e.g., different positions, angles, spacing, etc.) and multiple straps to facilitate modular and highly adjustable support configurations.

The various straps 22 can be constructed of an elastic material to provide additional tensioning adjustment. Again, the free end 22a of the strap 22 can include a hook and loop fastener (e.g., Velcro®), clips, fasteners, snaps, or like engagement features to retain the free end 22a securely in place to maintain the desired tensioning and support across the strap path. As shown in FIGS. 33 and 35, the free ends 22a can be extended or threaded through an aperture 29a of a clip device 29. The clip devices 29 can be constructed of various materials, including plastic or metals. The clip device 29 can further include an arm or member 29b adapted to engage with the various loop devices 60a, 60b, 60c, and the like. The member 29b can be constructed to include a degree of flex to facilitate attachment and accommodate various tensioning adjustment of the straps. Hook and loop fasteners 70 provided at the free ends 22a of the straps 22 can further facilitate adjustable securement of the free ends 22a to the apertures 29a upon threading the free ends 22a through the apertures 29a. In various embodiments, a clip device 29 can be provided (e.g., sewn or otherwise attached to) one end of the strap, while a second clip device 29 is separately provided and can be threaded though the second clip device 29 as described herein to promote selective connectivity to the loop devices. Selective placement and use of these loop devices provides highly targeted adjustability for tensioning the garment at desired regions to facilitate personalized and focused implant support and stabilization. The patient can work with a physician after the surgical procedure to attach and tension straps to specific loops such that only those loops needed to achieve the tensioning support, stabilization, angles and optimal positioning are used. Similarly, the patient or the patient's physician can later adjust the system by using different straps or by positioning and securing the straps at different areas or along different tensioning paths between opposing or nearby loops, as needed.

As depicted, in certain embodiments, certain of the straps 22 extending to the side loop device 60c can be measurably wider (FIG. 36), and longer, than the straps 22 extending generally vertically between opposing loop devices 60a.

The materials, structures, loops, perforations, rails, substrates and other devices disclosed herein can be employed at any portion of the garment 10 (e.g., front, back, sides) and can assume the construct of any of the disclosure herein without deviating from the spirit and scope of the present invention. Further, the portions, perforations, rails, substrates, and the like can be constructed of rigid, semi-rigid, flexible, and semi-flexible materials as desired for embodiments and particular applications of the present invention.

Certain embodiments of the garment 10 can include a combination of the rail devices 20, the rails 56 and substrates 50, and/or the loop devices 60a, 60b at various portions of the garment 10, depending on the particular modularity and adjustability desired for that portion.

While embodiments of the present invention have been described with a bra or like garment, the various configurations, devices, techniques and methods disclosed herein can be employed with other garments—e.g., swimsuits, shirts, support belts, and the like.

What is claimed is:

1. A compression garment system, comprising,
a first breast region;
a second breast region;
a plurality of first adjustment portions provided to the first breast region;
a plurality of second adjustment portions provided to the second breast region;
at least one first strap device having end attachment portions such that at least one of the end attachment portions of the at least one first strap device is selectively connected with at least one of the first adjustment portions to provide selective support for the first breast region; and
at least one second strap device having end attachment portions such that at least one of the end attachment portions of the at least one second strap devices is selectively connected with at least one of the second adjustment portions to provide selective support for the second breast region.

2. The system of claim 1, wherein at least one of the plurality of first adjustment portions includes an attachment rail.

3. The system of claim 1, wherein at least one of the plurality of first adjustment portions includes an attachment substrate.

4. The system of claim 3, wherein the attachment substrate is perforated.

5. The system of claim 1, wherein at least one of the plurality of first adjustment portions includes a loop device.

6. The system of claim 1, further including a first clip provided with the at least one first strap device to selectively connect with at least one of the plurality of first adjustment portions.

7. The system of claim 1, further including a side region including at least one side adjustment portion.

8. The system of claim 7, wherein the at least one side adjustment portion includes a side loop device such that the at least one first strap device is adapted to selectively connect between the side loop device and at least one of the plurality of first adjustment portions.

9. The system of claim 1, further including a second side region including at least one second side adjustment portion.

10. The system of claim 9, wherein the at least one second side adjustment portion includes a second side loop device such that the at least one second strap device is adapted to selectively connect between the second side loop device and at least one of the plurality of second adjustment portions.

11. The system of claim 1, further including a first clip provided with the at least one strap device to selectively connect with at least one of the plurality of first adjustment portions, wherein the first clip is generally six-shaped.

12. A compression garment system, comprising,
a first breast region;
a second breast region;
at least one side region;
at least one first loop adjustment portion provided to the first breast region;
at least one second loop adjustment portion provided to the second breast region;
at least one side loop adjustment portion provided to the at least one side region; and
at least one first strap device having first and second attachment portions such that the first loop attachment portion is selectively connectable with the at least one side loop adjustment portion.

13. The system of claim 12, further including a first clip provided with the at least one first strap device to selectively connect with the at least one first loop adjustment portion.

14. The system of claim 12, wherein the at least one side region includes first and second side regions.

15. The system of claim 14, wherein the second side region includes at least one second side loop portion.

16. The system of claim 15, further including at least one second strap device having first and second attachment portions such that the at least one second loop adjustment portion is operably connectable with the at least second side loop portion.

17. The system of claim 12, further including a first clip provided with the at least one first strap device to selectively connect with the at least one first loop adjustment portion, wherein the first clip is generally shaped as the number six.

18. The system of claim 12, further including a drainage device.

19. The system of claim 18, wherein the drainage device is adapted for operably connecting with one of the first and second breast regions.

20. The system of claim 12, further including a plurality of first clips, with one of the plurality of first clips selectively connectable with the at least one first loop adjustment portion, and another of the plurality of first clips selectively connectable with the at least one side loop adjustment portion.
